(19) United States
(12) Patent Application Publication
(10) Pub. No.: US 2007/0087013 A1
(43) Pub. Date: Apr. 19, 2007

(54) Orally-administered live bacterial vaccines for plague

(76) Inventors: Donata Sizemore, Grove City, OH (US); Steven A. Tingo, Lebanon, IL (US); Kevin P. Killeen, Milton, MA (US)

Correspondence Address: Leon R Yankwich;Yankwich & Associates, 201 Broadway-6th Floor, Cambridge, MA, 02139, US

(21) Appl. No.: 10/581,466

(22) PCT Filed: Dec. 9, 2004
(86) PCT No.: PCT/US04/41282

§ 371 Date: Jun. 1, 2006

Related U.S. Application Data
(60) Provisional application No. 60/528,140, filed on Dec. 9, 2003.
(60) Provisional application No. 60/559,259, filed on Apr. 2, 2004.
(60) Provisional application No. 60/573,517, filed on May 22, 2004.
(60) Provisional application No. 60/610,474, filed on Sep. 16, 2004.

(51) Int. Cl. A61K 39/02; C12N 1/21

(52) U.S. Cl. 424/200.1; 435/252.3

(57) ABSTRACT

The invention provides live, attenuated Salmonella bacterial strains that express one or more plague antigens of Yersinia pestis for use in live vaccine compositions that can be orally administered to an individual to protect against plague.

### CROSS-REFERENCES TO RELATED APPLICATIONS

This application claims priority to U.S. provisional application Nos. 60/528,140, filed Dec. 9, 2003; 60/559,259, filed Apr. 2, 2004; 60/573,517, filed May 22, 2004; and 60/610,474, filed Sep. 16, 2004.

### STATEMENT OF GOVERNMENTAL INTEREST

The work leading to the invention described herein was partly funded by the United States Department of Defense. Accordingly, the Federal Government has certain rights in the invention.

### FIELD OF THE INVENTION

This invention is generally in the field of live bacterial vaccines. In particular, this invention relates to live attenuated bacterial strains vectoring plague antigens that can be administered orally to an individual to elicit an immune response to protect the individual from plague.

### BACKGROUND OF THE INVENTION

Plague is caused by the Gram-negative bacterium, Yersinia pestis. Among the oldest documented infectious diseases, plague has caused multiple epidemics and at least three pandemics throughout recorded history. Plague usually manifests in humans in bubonic (infection of lymph nodes) or pneumonic (infection of lungs) forms, but may also spread to the blood resulting in a septicemic form of the disease. Bubonic plague typically results from the bite of a flea infected with Y. pestis bacteria, whereas pneumonic plague may be initiated by intimate contact and inhalation of contaminated nasal and airborne droplets from a patient or infected animal. The clinical presentation of bubonic plague is a very painful, usually swollen, hemorrhagic, necrotic, and often hot-to-the touch lymph node, called a bubo. Onset of bubonic plague is usually 2 to 6 days after a person is exposed to (infected with) the plague bacillus. The incubation period of primary pneumonic plague is 1 to 3 days and is characterized by development of an overwhelming pneumonia with high fever, cough, bloody sputum, and chills. The mortality rates for plague are staggering. In untreated cases of bubonic plague there is a 40%-60% mortality rate, and in the case of pneumonic plague, the mortality is 100% for patients not treated within the first 24 hours of infection. A primary septicemic plague may also occur when the infecting plague bacillus bypasses the lymph nodes and proliferates in the circulatory system. If left untreated, the mortality rate of septicemic plague is 100%.

In the United States an average of approximately 10 to 20 cases of plague are reported annually. Worldwide, there are approximately 1,000 to 2,000 cases reported each year. Approximately half of all reported cases are in persons under 20 years of age. During the 1980s, epidemic plague occurred each year in Africa, Asia, or South America. Almost all of the cases reported during the decade occurred among people living in small rural towns, villages, or agricultural areas. In the early 1990s, outbreaks of plague also occurred in East African countries, Madagascar, Peru, and India (Dennis and Hughes, N. Eng. J. Med., 337(10):702-704 (1997)). Plague epidemics are generally associated with human contact with rats carrying fleas infected with Y. pestis, although, other rodents infested with infected fleas may serve as reservoirs of the disease as well. For example, in the Southwestern United States, “sylvatic” plague may result from transmission of plague bacteria to humans by the bite of infected fleas populating a variety of rodents, including ground squirrels, prairie dogs, marmots, mice, and tree squirrels.

If administered sufficiently early, a number of antibiotics (e.g., streptomycin, chloramphenicol, tetracycline), alone or in combination, can be effective against plague. Antibiotics (especially tetracycline and sulfonamides) may also be administered prophylactically to any individual that is presumed to be at risk for plague, e.g., anyone suspected of contacting infected individuals or animals. However, reliance on treating plague with antibiotics clearly presents a number of problems. For example, rural and underdeveloped areas of the world may lack access to sufficient stocks of effective antibiotics and/or the skilled personnel needed to administer the antibiotics to treat patients and prevent a plague epidemic. Moreover, in recent years, strains of plague bacteria have emerged that are resistant to one or more of the antibiotics traditionally employed to treat patients, and such resistance has been found to be encoded on transmissible plasmids (see, e.g., Galimand et al., N. Eng. J. Med., 337(10):677-680 (1997); Dennis and Hughes, N. Eng. J. Med., 337(10):702-704 (1997)).

A vaccine for plague that is easily administered and that provides immunity for a reasonable duration (e.g., months to years) would clearly be preferred over the current dependency on antibiotics. A former injectable vaccine employing killed Y. pestis that provided some immunity to plague is no longer commercially available in the United States. Such previous vaccines were administered parenterally, which principally elicits production of systemic antibody (immunoglobulin G, IgG), but not mucosal antibody (secretory IgA). Such mucosal immunity to plague is particularly desirable to protect against the pneumonic form of the disease. More recently, pre-clinical immunogenicity and efficacy studies evaluating candidate plague vaccines that were based on immunization with Y. pestis F1 capsule and/or V antigens have demonstrated that serum IgG is a reliable correlate of protection against intravenous challenge with Y. pestis, although T-lymphocyte responses may also contribute to protective immunity in experimental animals (Williamson et al., Clin. Exp. Immunol., 116:107-114 (1999); Titball and Williamson, Vaccine, 19:4175-4184 (2001)). Such candidate plague vaccines have all required a multi-dose injection regimen and have not provided reliable protection against the pneumonic form of the disease (Titball and Williamson, Vaccine, 19:4175-4184 (2001)).

In addition to the need for more effective treatments for plague as it naturally occurs in diverse areas of the world, there is also the concern that Y. pestis has long been recognized as a possible agent for biological warfare and, more recently, as a candidate agent for a weapon of bioterrorism (see, e.g., Inglesby et al., J. Am. Med. Assoc., 283(17):2281-2290 (2000); see, also, http://sis.nlm.nih.gov/Tox/biologicalwarfare.htm). The technology required to handle, grow, contain, and maintain even lethal bacterial pathogens such as Y. pestis is relatively low cost and requires easily taught microbiological techniques that are routinely employed for handling any strain of pathogenic bacteria. Moreover, even a relatively crude bacteria-based weapon device might be sufficient for the purpose of bioterrorism. For example, as noted above, in the case of pneumonic plague the lethal route of infection by the Y. pestis pathogen initiates at the mucosal surface of the respiratory tract. Thus, even a relatively modest device that disperses an aerosol of the plague pathogen into a relatively small population or group of individuals might result in considerable suffering and widespread panic. The extent of such a scenario could be greatly limited by the availability of an effective plague vaccine that can be easily produced and rapidly administered not only to infected individuals but also to healthcare providers and other “first responders” (i.e., various civil and military emergency personnel) that must serve in the vicinity of a terrorist incident or disease outbreak.

Titball et al. (U.S. Pat. No. 5,985,285) previously described potential vaccines for plague comprising recombinant forms of Y. pestis F1 and V protein antigens, including a live vaccine of an attenuated strain of Salmonella typhimurium (i.e., S. enterica serovar Typhimurium) that expressed a recombinant fusion protein comprising F1 and V antigen polypeptides and that provided protection against challenge in mice. However, it has become apparent that certain assumptions, statements, and experimental designs described by Titball et al. regarding live plague vaccines would be too general and/or too hazardous to provide a live vaccine for plague that would be acceptable for use in humans. For example, Titball et al. state that any of a variety of known strains of Salmonella bacteria that have an attenuated virulence may be genetically engineered and employed as live bacterial carriers (bacterial vectors) that express Y. pestis F1 and V antigen polypeptides to elicit an immune response for plague, including attenuated strains of S. typhimurium and, for use in humans, attenuated strains of S. typhi (i.e., S. enterica serovar Typhi; see, e.g., col. 2, line 66-col. 3, line 31, of Titball et al.). In support of such broad teaching, Titball et al. describe the construction of a bacterial strain of S. typhimurium that was attenuated by a deletion mutation in the aroA gene (a gene required for synthesis of aromatic compounds such as aromatic amino acids) and that carried a multi-copy expression plasmid that encoded a recombinant F1-V fusion protein and a selectable ampicillin resistance marker (see, e.g., col. 11-col. 18, of Titball et al.). Animals (mice) that were injected intravenously with the attenuated, recombinant S. typhimurium strain (vaccine strain) were also administered a subcutaneous dose of the antibiotic ampicillin to provide a selection in vivo for S. typhimurium bacteria that maintained the recombinant plasmid encoding the F1-V fusion protein (see, col. 17, lines 35-62, of Titball et al.). Mice were challenged with subcutaneously administered Y. pestis bacteria 57 days after receiving the vaccine strain and ampicillin. Most (i.e., 6 out of 7) of the animals that were injected with the live vaccine strain and ampicillin survived challenge with Y. pestis, whereas none (i.e., 0 out 5) of the control (no vaccine) animals survived challenge with Y. pestis (see, col. 18, lines 42-66, of Titball et al.).

However, it is now understood that, contrary to Titball et al., a live vaccine for use in humans cannot be any known attenuated Salmonella strain. In particular, Salmonella bacteria attenuated by mutations in aro genes induce undesirable reactions (i.e., are “reactogenic”) in humans. For example, aro mutants of S. typhi are not sufficiently attenuated in virulence, but retain the ability to pass from the gut into the bloodstream resulting in bacteremia (see, e.g., Hone et al., J. Clin. Invest., 90(2):412-420 (1992); Dilts et al., Vaccine, 18(15):1473-1484 (2000)). Thus, contrary to Titball et al., Salmonella strains that are attenuated only by an aro mutation could not be administered intravenously, intraperitoneally, subcutaneously, or even orally into humans as such strains would undoubtedly lead to a bacteremia and/or bacterial lipopolysaccharide (LPS)-induced shock (see, e.g., Hopf et al., Am. J. Emerg. Med., 2(1):13-19 (1984)). Thus, an intravenous injection of a live attenuated aroA mutant strain of S. typhimurium or S. typhi as described by Titball et al. does not demonstrate an acceptable oral live vaccine for use in humans. Furthermore, it is clear that in addition to the practical need for a vaccine that is easily administered to humans, the U.S. Food and Drug Administration and other public health agencies throughout the world would not permit the use of a live vaccine that depends on administration and maintenance of adequate intracellular levels of antibiotics to provide an in vivo selective pressure for the desired (i.e., antigen-expressing) form of a bacterial vaccine strain as employed by Titball et al.

In addition to the above, Titball et al. merely asserts to have provided the art with live plague vaccines that can be administered orally to humans (see, e.g., col. 1, lines 8-11; col. 3, lines 11-19, of Titball et al.) and that such vaccines elicit a mucosal immunity, which includes production of secretory anti-plague IgA response in the respiratory tract, which would be especially important for providing immunity against the deadly pneumonic plague (see, e.g., col. 2, lines 55-62, of Titball et al.). Although such mucosal immunity is a desirable feature of an orally administered live vaccine, Titball et al. provides no data that demonstrated immunity along the tissues of the mucosa of the animals. Accordingly, although a live vaccine that can establish a mucosal immunity against plague is highly desirable, such a vaccine that would be acceptable for use in humans is not provided by Titball et al.

The above comments illustrate that Titball et al. have not provided the field with an effective vaccine against plague. Clearly, needs remain for an effective, orally administered vaccine against plague.

### SUMMARY OF THE INVENTION

The invention described herein addresses the above problems, including the deficiencies of Titball et al. (U.S. Pat. No. 5,985,285), by providing live attenuated strains of serovars of Salmonella enterica that express one or more immunogenic polypeptide antigens of the plague bacillus Yersinia pestis. The Salmonella strains of the invention are attenuated by a mutation at a genetic locus other than a gene involved in the synthesis of aromatic compounds (aro) and other than by a single attenuating mutation in a gene for galactose utilization (e.g., the galE gene), either of which, alone, provides insufficient attenuation, and thereby avoiding a number of unacceptable side effects such as typhoid, septicemia, severe diarrhea, high fever, and shock. Accordingly, the attenuated Salmonella strains described herein are useful as live bacterial vaccines that can be orally administered to an individual to provide immunity to plague bacteria and, thereby, protection from plague.

In one embodiment, the invention provides a live vaccine composition for protecting against plague comprising a live attenuated bacterium that is a serovar of Salmonella enterica comprising:
an attenuating mutation in a genetic locus of the chromosome of said bacterium that attenuates virulence of said bacterium and wherein said attenuating mutation is not a single mutation in a gene that encodes a protein that is essential for the synthesis of an aromatic compound and is not a single mutation in a gene for galactose utilization;
a lethal mutation in a genetic locus in the chromosome of said bacterium wherein said lethal mutation prevents expression from said genetic locus of a protein that has an activity that is essential for synthesis of diaminopimelic acid (DAP);
an antigen-expressing, multi-copy plasmid comprising:
   a nucleotide sequence coding for an immunogenic polypeptide comprising a Yersinia pestis V antigen, an immunogenic portion of said V antigen, a Yersinia pestis F1 antigen, an immunogenic portion of said F1 antigen, or a combination thereof, wherein said nucleotide sequence is operably linked to a promoter that permits intracellular expression of said immunogenic polypeptide from said plasmid,
   a gene encoding a protein that has an activity that is essential for synthesis of diaminopimelic acid (DAP), wherein expression of said protein is essential for DAP synthesis and complements said lethal mutation in the chromosome of said bacterium and thereby permits growth of said bacterium in the absence of exogenously supplied DAP, and
   an origin of replication that permits multiple copies of said plasmid to be maintained in said bacterium,
      wherein said live vaccine composition elicits an immune response to one or more Yersinia pestis antigens when administered orally to an individual.

An attenuating mutation useful in the Salmonella bacterial strains described herein may be in a genetic locus selected from the group consisting of phoP, phoQ, cdt, cya, crp, poxA, rpoS, htrA, nuoG, pmi, pabA, pts, damA, purB, gua, cadA, rfc, rjb, rfa, ompR, and combinations thereof.

A particularly useful mutation for attenuating virulence of the Salmonella strains of the vaccine compositions of the invention is a deletion that inactivates the phoP and phoQ genetic loci (ΔphoP/Q) on the Salmonella chromosome.

A particularly useful lethal mutation for use in the Salmonella strains of the vaccine composition of the invention is a deletion in the asdA gene (ΔasdA) of the Salmonella chromosome.

The serovars of S. enterica that may be used as the attenuated bacterium of the live vaccine compositions described herein include, without limitation, Salmonella enterica serovar Typhimurium (“S. typhimurium”), Salmonella enterica serovar Typhi (“S. typhi”), Salmonella enterica serovar Paratyphi B (“S. paratyphi B”), Salmonella enterica serovar Paratyphi C (“S. paratyphi C”), Salmonella enterica serovar Hadar (“S. hadar”), Salmonella enterica serovar Enteriditis (“S. enteriditis”), Salmonella enterica serovar Kentucky (“S. kentucky”), Salmonella enterica serovar Infantis (“S. infantis”), Salmonella enterica serovar Pullorum (“S. pullorum”), Salmonella enterica serovar Gallinarum (“S. gallinarum”), Salmonella enterica serovar Muenchen (“S. muenchen”), Salmonella enterica serovar Anatum (“S. anatum”), Salmonella enterica serovar Dublin (“S. dublin”), Salmonella enterica serovar Derby (“S. derby”), and Salmonella enterica serovar Choleraesuis var. kunzendorf (“S. cholerae kunzendorf”).

In a preferred embodiment, the invention provides live vaccine compositions comprising one or more of the following strains of S. enterica serovar Typhimurium (“S. typhimurium”) as deposited with the American Type Culture Collection (“ATCC”, 10801 University Blvd., Manassas, Va., 20110-2209 USA) under the terms of the Budapest Treaty on Dec. 2, 2004: S. typhimurium strain M020 (ATCC Accession No. PTA-6406), S. typhimurium M022 (ATCC Accession No. PTA-6407), S. typhimurium M023 (ATCC Accession No. PTA-6408), S. typhimurium M048 (ATCC Accession No. PTA-6409), and S. typhimurium M049 (ATCC Accession No. PTA-6410).

The live vaccine compositions are suitable for oral administration to an individual to provide protection from plague. Preferably, a vaccine composition comprises a suspension of a live bacterial strain described herein in a physiologically accepted buffer or saline solution that can be swallowed from the mouth of an individual. However, oral administration of a vaccine composition to an individual may also include, without limitation, administering a suspension of a bacterial vaccine strain described herein through a nasojejunal or gastrostomy tube and administration of a suppository that releases a live bacterial vaccine strain to the lower intestinal tract of an individual.

### BRIEF DESCRIPTION OF THE DRAWINGS

FIG. 1 shows a diagram of the 4178 base pair (bp), antigen-expressing plasmid pMEG-1621, including relative locations of major genetic loci and restriction endonuclease sites. Numbers after names of restriction endonucleases indicate specific restriction sites in the plasmid. “Ptrc” and bold arrow refer to a functional trc promoter operably linked to a structural coding sequence for an F1-V antigen fusion polypeptide. “asd” is a functional, wildtype bacterial gene that encodes a functional aspartate semialdehyde dehydrogenase. “pBR ori” refers to the origin of replication from plasmid pBR322. “5S T1 T2” refers to the T1 and T2 transcriptional terminators of the 5S bacterial ribosomal RNA gene. Arrows indicate direction of transcription. See text for details.

FIG. 2 shows a diagram of the 3006 base pair (bp), antigen-expressing plasmid pMEG-1707, including relative locations of major genetic loci and restriction endonuclease sites. Numbers after names of restriction endonucleases indicate specific restriction sites in the plasmid. “Ptrc” and bold arrow refer to a functional trc promoter operably linked to a structural coding sequence for an F1 antigen polypeptide. “asd” is a functional, wildtype bacterial gene that encodes a functional aspartate semialdehyde dehydrogenase. “pUC18 ori” refers to the origin of replication from plasmid pUC18. “5S T1 T2” refers to the T1 and T2 transcriptional terminators of the 5S bacterial ribosomal RNA gene. Arrows indicate direction of transcription. See text for details.

FIG. 3 shows a diagram of the 3738 base pair (bp), antigen-expressing plasmid pMEG-1692, including relative locations of major genetic loci and restriction endonuclease sites. Numbers after names of restriction endonucleases indicate specific restriction sites in the plasmid. “Ptrc” and bold arrow refer to a functional trc promoter operably linked to a structural coding sequence for a V antigen polypeptide. “asd” is a functional, wildtype bacterial gene that encodes a functional aspartate semialdehyde dehydrogenase. “pBR ori” refers to the origin of replication from plasmid pBR322. “5S T1 T2” refers to the T1 and T2 transcriptional terminators of the 5S bacterial ribosomal RNA gene. Arrows indicate direction of transcription. See text for details.

FIG. 4 shows a diagram of the 4203 base pair (bp), antigen-expressing plasmid pMEG-1967, including relative locations of major genetic loci and restriction endonuclease sites. Numbers after names of restriction endonucleases indicate specific restriction sites in the plasmid. “Ptrc” and bold arrow immediately above refer to a functional trc promoter operably linked to a structural coding sequence for an F1 antigen polypeptide linked in frame to a structural coding sequence for V antigen polypeptide. In pMEG-1967, Ptrc directs transcription of a single messenger RNA (mRNA) encoding separate F1 and V antigen polypeptides. “RBS” indicates the presence of a separate ribosome-binding site for separate translation of the V antigen polypeptide from the single mRNA transcript. “asd” is a functional, wildtype bacterial gene that encodes a functional aspartate semialdehyde dehydrogenase. “pBR ori” refers to the origin of replication from plasmid pBR322. “5S T1 T2” refers to the T1 and T2 transcriptional terminators of the 5S bacterial ribosomal RNA gene. Arrows indicate direction of transcription. See text for details.

FIG. 5 shows a diagram of the 4010 base pair (bp), antigen-expressing plasmid pMEG-1968, including relative locations of major genetic loci and restriction endonuclease sites. Numbers after names of restriction endonucleases indicate specific restriction sites in the plasmid. “Ptrc” and bold arrow immediately above refer to a functional trc promoter operably linked to a structural coding sequence for an F1 antigen polypeptide linked in frame to a structural coding sequence for V antigen polypeptide. In pMEG-1968, Ptrc directs transcription of a single messenger RNA (mRNA) encoding separate F1 and V antigen polypeptides. “RBS” indicates the presence of a separate ribosome-binding site for separate translation of the V antigen polypeptide from the single mRNA transcript. “asd” is a functional, wildtype bacterial gene that encodes a functional aspartate semialdehyde dehydrogenase. “pUC18 ori” refers to the origin of replication from plasmid pUC18. “5S T1 T2” refers to the T1 and T2 transcriptional terminators of the 5S bacterial ribosomal RNA gene. Arrows indicate direction of transcription. See text for details.

### DETAILED DESCRIPTION OF THE INVENTION

In order that the invention may be more fully understood, the following terms are defined.

As used herein, “attenuated”, “attenuation”, and similar terms refer to elimination or reduction of the natural virulence of a bacterium in a particular host organism, such as a mammal. “Virulence” is the degree or ability of a pathogenic microorganism to produce disease in a host organism. A bacterium may be virulent for one species of host organism (e.g., a mouse) and not virulent for another species of host organism (e.g., a human). Hence, broadly, an “attenuated” bacterium or strain of bacteria is attenuated in virulence toward at least one species of host organism that is susceptible to infection and disease by a virulent form of the bacterium or strain of the bacterium.

As used herein, the term “genetic locus” is a broad term and comprises any designated site in the genome (the total genetic content of an organism) or in a particular nucleotide sequence of a chromosome or replicating nucleic acid molecule (e.g., a plasmid), including but not limited to a gene, nucleotide coding sequence (for a protein or RNA), operon, regulon, promoter, regulatory site (including transcriptional terminator sites, ribosome binding sites, transcriptional inhibitor binding sites, transcriptional activator binding sites), origin of replication, intercistronic region, and portions therein. A genetic locus may be identified and characterized by any of a variety of in vivo and/or in vitro methods available in the art, including but not limited to, conjugation studies, crossover frequencies, transformation analysis, transfection analysis, restriction enzyme mapping protocols, nucleic acid hybridization analyses, polymerase chain reaction (PCR) protocols, nuclease protection assays, and direct nucleic acid sequence analysis.

As used herein, the term “infection” has the meaning generally used and understood by persons skilled in the art and includes the invasion and multiplication of a microorganism in or on a host organism (“host”, “individual”, “patient”) with or without a manifestation of a disease (see, “virulence” above). Infectious microorganisms include pathogenic bacteria, such as Yersinia pestis, that can cause serious diseases when infecting an unprotected individual. An infection may occur at one or more sites in or on an individual. An infection may be unintentional (e.g., unintended ingestion, inhalation, contamination of wounds, etc.) or intentional (e.g., administration of a live vaccine bacterial strain, experimental challenge with a pathogenic bacterial strain). In a vertebrate host organism, such as a mammal, a site of infection includes, but is not limited to, the respiratory system, the alimentary canal (gut), the circulatory system, the skin, the endocrine system, the neural system, and intercellular spaces. Some degree and form of replication or multiplication of an infective microorganism is required for the microorganism to persist at a site of infection. However, replication may vary widely among infecting microorganisms. Accordingly, replication of infecting microorganisms comprises, but is not limited to, persistent and continuous multiplication of the microorganisms and transient or temporary maintenance of microorganisms at a specific location. Whereas “infection” of a host organism by a pathogenic microorganism is undesirable owing to the potential for causing disease in the host, an “infection” of a host individual with a live vaccine comprising genetically altered, attenuated Salamonella bacterial strain as described herein is desirable because of the ability of the bacterial strain to elicit a protective immune response to antigens of Y. pestis bacteria that cause plague in humans and other mammals.

As used herein, the terms “disease” and “disorder” have the meaning generally known and understood in the art and comprise any abnormal condition in the function or well being of a host individual. A diagnosis of a particular disease or disorder, such as plague, by a healthcare professional may be made by direct examination and/or consideration of results of one or more diagnostic tests.

A “live vaccine composition”, “live vaccine”, “live bacterial vaccine”, and similar terms refer to a composition comprising a strain of live Salmonella bacteria that expresses at least one antigen of Y. pestis, e.g., the F1 antigen, the V antigen, or a combination thereof, such that when administered to an individual, the bacteria will elicit an immune response in the individual against the plague antigen(s) expressed in the Salmonella bacteria and, thereby, provide at least partial protective immunity against plague. Such protective immunity may be evidenced by any of a variety of observable or detectable conditions, including but not limited to, diminution of one or more disease symptoms (e.g., fever, pain, diarrhea, bleeding, inflammation of lymph nodes, weakness, malaise), shorter duration of illness, diminution of tissue damage, regeneration of healthy tissue, clearance of pathogenic microorganisms from the individual, and increased sense of well being by the individual. Although highly desired, it is understood by persons skilled in the art that no vaccine is expected to induce complete protection from a disease in every individual that is administered the vaccine or that protective immunity is expected to last throughout the lifetime of an individual without periodic “booster” administrations of a vaccine composition. It is also understood that a live vaccine comprising a bacterium described herein may be, at the discretion of a healthcare professional, administered to an individual who has not presented symptoms of plague but is considered to be at risk of infection or is known to already have been exposed to Y. pestis bacteria, e.g., by proximity or contact with plague patients or bacterially contaminated air, liquids, or surfaces.

The terms “oral”, “enteral”, “enterally”, “orally”, “non-parenteral”, “non-parenterally”, and the like, refer to administration of a compound or composition to an individual by a route or mode along the alimentary canal. Examples of “oral” routes of administration of a vaccine composition include, without limitation, swallowing liquid or solid forms of a vaccine composition from the mouth, administration of a vaccine composition through a nasojejunal or gastrostomy tube, intraduodenal administration of a vaccine composition, and rectal administration, e.g., using suppositories that release a live bacterial vaccine strain described herein to the lower intestinal tract of the alimentary canal.

The term “recombinant” is used to describe non-naturally altered or manipulated nucleic acids, cells transformed, electroporated, or transfected with exogenous nucleic acids, and polypeptides expressed non-naturally, e.g., through manipulation of isolated nucleic acids and transformation of cells. The term “recombinant” specifically encompasses nucleic acid molecules that have been constructed, at least in part, in vitro using genetic engineering techniques, and use of the term “recombinant” as an adjective to describe a molecule, construct, vector, cell, polypeptide, or polynucleotide specifically excludes naturally existing forms of such molecules, constructs, vectors, cells, polypeptides, or polynucleotides.

As used herein, the term “salmonella” (plural, “salmonellae”) and “Salmonella” refers to a bacterium that is a serovar of Salmonella enterica. A number of serovars of S. enterica are known. Particularly preferred salmonella bacteria useful in the invention are attenuated strains of Salmonella enterica serovar Typhimurium (“S. typhimurium”) and serovar Typhi (“S. typhi”) as described herein.

As used herein, the terms “strain” and “isolate” are synonymous and refer to a particular isolated bacterium and its genetically identical progeny. Actual examples of particular strains of bacteria developed or isolated by human effort are indicated herein by specific letter and numerical designations (e.g. strains M020, M022, M023, M048, M049).

The definitions of other terms used herein are those understood and used by persons skilled in the art and/or will be evident to persons skilled in the art from usage in the text.

This invention provides live vaccine compositions for protecting against plague comprising live Salmonella enterica serovars that are genetically engineered to express one or more plague antigen polypeptides, such as the F1 and V antigens of Yersinia pestis. Salmonella bacteria have been recognized as being particularly useful as live “host” vectors for orally administered vaccines because these bacteria are enteric organisms that, when ingested, can infect and persist in the gut (especially the intestines) of humans and animals. Accordingly, when orally administered to an individual, live Salmonella bacteria that are genetically engineered to express one or more plague antigens as described herein have the inherent ability to establish a population (infection) in the gut and, thereby, provide a desirable source of immunogenic plague antigen polypeptide(s) to elicit an immune response in the mucosal tissue of the individual. As a variety of Salmonella bacteria are known to be highly virulent to most hosts, e.g., causing typhoid fever or severe diarrhea in humans and other mammals, the virulence of Salmonella bacterial strains toward an individual that is targeted to receive a vaccine composition must be attenuated. Attenuation of virulence of a bacterium is not restricted to the elimination or inhibition of any particular mechanism and may be obtained by mutation of one or more genes in the Salmonella genome (which may include chromosomal and non-chromosomal genetic material). Thus, an “attenuating mutation” may comprise a single site mutation or multiple mutations that may together provide a phenotype of attenuated virulence toward a particular host individual who is to receive a live vaccine composition for plague.

In recent years, a variety of bacteria and, particularly, serovars of Salmonella enterica, have been developed that are attenuated for pathogenic virulence in an individual (e.g., humans or other mammals), and thus proposed as useful for developing various live bacterial vaccines (see, e.g., U.S. Pat. Nos. 5,389,368; 5,468,485; 5,387,744; 5,424,065; Zhang-Barber et al., Vaccine, 17; 2538-2545 (1999); all incorporated herein by reference). In the case of strains of Salmonella, mutations at a number of genetic loci have been shown to attenuate virulence including, but not limited to, the genetic loci phoP, phoQ, cdt, cya, crp, poxA, rpoS, htrA, nuoG, pmi, pabA, pts, damA, purB, gua, cadA, rfc, rjb, rfa, ompR, and combinations thereof. However, not every attenuating mutation is acceptable for use as a live vaccine according to the invention (see, e.g., review in Kotton and Hohmann, Infect. Immun., 72(10):5535-5547 (2004)). For example, mutations in the aro genes of the Salmonella genome only partially attenuate virulence, allowing Salmonella bacteria to pass from the gut into the bloodstream as recently observed for aro mutants of S. typhi (see, e.g., Hone et al., J. Clin. Invest., 90(2):412-420 (1992); Dilts et al., Vaccine, 18(15):1473-11484 (2000)). Thus, Salmonella strains attenuated only by an aro mutation are not suitable for administration to humans by any route (i.e., parenteral or oral) as such partially attenuated strains would most likely result in a life-threatening bacteremia and/or bacterial lipopolysaccharide (LPS)-induced shock (see, e.g., Hopf et al., Am. J. Emerg. Med., 2(1):13-19 (1984)). In addition, mutations in galE (a gene involved in galatose utilization) have been shown to provide insufficient attenuation of S. typhi for use in humans (see, e.g., Hone et al., Infect. Immun., 56:1326-1333 (1988)).

By way of example, live plague vaccines of the invention include strains of S. enterica serovar Typhimurium (S. typhimurium) that are attenuated in virulence by mutation in the phoP and phoQ loci on the Salmonella bacterial chromosome (see, e.g., DiPetrillo et al., Vaccine, 18:449-459 (1999); Angelakopolous and Hohmann, Infect. Immun., 68(4):2135-2141 (2000)). A preferred attenuating mutation for use in the strains of the invention is a deletion mutation of a region of deoxyribonucleic acid (DNA) that traverses two contiguous genetic loci, i.e., phoP and phoQ, on the Salmonella chromosome (referred to variously as “phoP/phoQ-deleted”, “ΔphoP/Q”, “ΔphoPQ”, “ΔphoP ΔphoQ”, “ΔphoP/ΔphoQ”). The Salmonella phoP locus is a bacterial regulon comprised of two contiguous genes, phoP and phoQ. Response to environmental signals by phoP is coordinated by the cytoplasmic transcriptional regulator, PhoP, and the membrane-associated sensor kinase, PhoQ (Miller et al., Proc. Natl. Acad. Sci. USA, 86(13):5054-5058 (1989); Groisman et al., Proc. Natl. Acad. Sci. USA, 86(18):7077-7081 (1989)). PhoP and PhoQ regulate a series of unlinked genes that have been classified as PhoP-activated genes (pags) and PhoP-repressed genes (prgs). The regulation of these two classes of genes has been shown to play a role in the Salmonella defensin resistance, survival in macrophages, and acid sensitivity (see, e.g., Miller et al., Proc. Natl. Acad. Sci. USA, 86(13):5054-5058 (1989); Miller et al., Infect. Immun., 58(11):3706-3710 (1990); Fields et al., Science, 243(4894):1059-1062 (1989); Galan and Curtiss, Microb. Pathog., 6(6):433-443 (1989); Foster and Hall, J. Bacteriol., 172(2):771-778 (1990)). PhoP has also been shown to be essential for mouse virulence in the S. typhimurium mouse typhoid fever model (Miller et al., Proc. Natl. Acad. Sci. USA, 86(13):5054-5058 (1989); Miller and Mekalanos, J. Bacteriol., 172(2):2485-2490 (1990)).

Although not wishing to be bound by any particular mechanism, an effective mucosal immune response to plague antigen(s) in humans by oral administration of phoP/Q mutant, attenuated strains of S. typhimurium as described herein may be due to the ability of such mutant strains to persist in the intestinal tract longer than other Salmonella strains that have been attenuated by mutation at one or more other genetic loci and/or because such phoP/Q mutant strains of S. typhimurium may also provide greater stability for the plague antigen-expressing plasmids that reside in the vaccine strains described herein.

Each bacterial strain useful in the invention carries an antigen-expressing plasmid that encodes and directs expression of one or more plague antigens of Yersinia pestis when resident in an attenuated Salmonella strain described herein. As noted above, plague antigens that are particularly useful in the invention include an F1 antigen polypeptide (or immunogenic portion thereof), a V antigen polypeptide (or immunogenic portion thereof), and a fusion polypeptide comprising an F1 polypeptide (or immunogenic portion thereof) linked in-frame to a V polypeptide (or immunogenic portion thereof) (see, e.g., Miller et al., FEMS Immunol. Med. Microbiol., 21:213-221 (1998); Williamson et al., FEMS Immunol. Med. Microbiol., 12:223-230 (1995); Heath et al., Vaccine, 16(11-12):1131-1137 (1998)). An example of a nucleotide sequence that encodes an F1 antigen polypeptide of Y. pestis that may be used in the invention has the nucleotide coding sequence of SEQ ID NO:1, and the corresponding encoded F1 polypeptide has the amino acid sequence of SEQ ID NO:2. An example of a nucleotide sequence that encodes a V antigen polypeptide of Y. pestis that may be used in the invention has the nucleotide coding sequence of SEQ ID NO:3, and the corresponding encoded V antigen polypeptide has shown the amino acid sequence of SEQ ID NO:4. By way of further example, a nucleotide sequence that encodes an F1-V fusion polypeptide useful in the invention has the nucleotide coding sequence of SEQ ID NO:5, and the corresponding encoded F1-V fusion polypeptide has the amino acid sequence of SEQ ID NO:6.

Preferably, antigen-expressing plasmids useful in the invention are engineered to express a plague antigen polypeptide intracellularly in a host Salmonella strain. Accordingly, plague antigen polypeptides expressed from antigen-expressing plasmids in the vaccine strains described herein, are preferably not linked to a signal peptide or other peptide for membrane localization or secretion across the cell membrane.

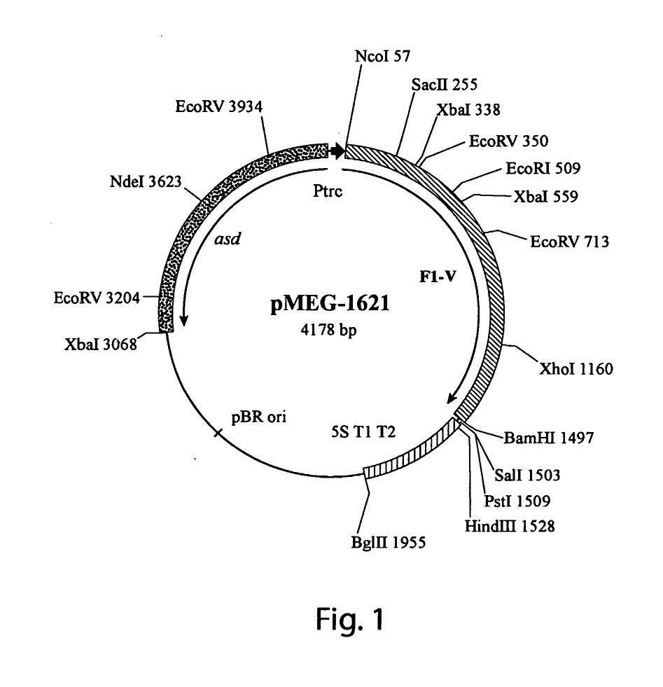

An antigen-expressing plasmid in the bacterial strains described herein may also contain one or more transcriptional terminators adjacent to the 3′ end of a particular nucleotide sequence on the plasmid to prevent undesired transcription into another region of the plasmid. Such transcription terminators thus serve to prevent transcription from extending into and potentially interfering with other critical plasmid functions, e.g., replication or gene expression. Examples of transcriptional terminators that may be used in the antigen-expressing plasmids described herein include, but are not limited to, the T1 and T2 transcription terminators from 5S ribosomal RNA bacterial genes (see, e.g., FIGS. 1-5; Brosius and Holy, Proc. Natl. Acad. Sci. USA, 81:6929-6933 (1984); Brosius, Gene, 27(2):161-172 (1984); Orosz et al., Eur. J. Biochem., 201(3):653-659 (1991)).

The expression plasmids are maintained in an attenuated bacterial host strain by employing the balanced lethal system based on complementation of a mutation in the chromosomal gene asd as previously described by Nakayama et al. (Bio/Technology, 6:693-697 (1988)). In this system, the attenuated strains of S. tyhphimurium carry a lethal mutation in the chromosomal gene for aspartate semialdehyde dehydrogenase (asd), which is required for synthesis of the cell wall component diaminopimelic acid (DAP). Absence of DAP leads to “DAPless” death and cell lysis of the asd mutant strains. The antigen-expressing plasmids carried by bacterial strains described herein carry a functional asd gene that encodes a functional aspartate semialdehyde dehydrogenase to complement the Asd⁻ phenotype of the host Salmonella bacterial strains, thereby providing an internal selective pressure for maintaining the antigen-expressing plasmid when the Salmonella strains are placed in an environment that lacks DAP, as in the case of the gut of humans and other mammals. Thus, an advantage to using this balanced lethal (complementation) system for maintaining the antigen-expressing plasmid in a live bacterial host is that it eliminates completely the dependence on a plasmid-encoded antibiotic resistance marker and the administration of the corresponding antibiotic to an individual in order to provide selective pressure in vivo for maintenance of the antigen-expressing plasmid in the bacterial strain (cf., Titball et al., above).

The antigen-expressing plasmids described herein comprise one or more nucleotide sequences that encode one or more polypeptides that, in turn, comprise one or more plague antigens, such as the F1 and V polypeptide antigens, or immunogenic portions thereof, from Yersinia pestis. Such coding sequences are operably linked to a promoter of transcription that functions in a Salmonella bacterial strain even when such a bacterial strain is ingested, i.e., when a live vaccine composition described herein is administered orally to an individual. A variety of naturally occurring, recombinant, and semi-synthetic promoters are known to function in enteric bacteria, such as Escherichia coli and serovars of S. enterica (see, e.g., Dunstan et al., Infect. Immun., 67(10):5133-5141 (1999)). Promoters (P) that are useful in the invention include, but are not limited to, well known and widely used promoters for gene expression such as the naturally occurring Plac of the lac operon and the semi-synthetic Ptrc (see, e.g., Amman et al., Gene, 25 (2-3):167-178 (1983)) and Ptac (see, e.g., Amann et al., Gene, 69(2):301-315 (1988)), as well as PpagC (see, e.g., Hohmann et al., Proc. Natl. Acad. Sci. USA, 92:2904-2908 (1995)), PpmrH (see, e.g., Gunn et al., Infect. Immun., 68:6139-6146 (2000)), PpmrD (see, e.g., Roland et al., J. Bacteriol., 176:3589-3597 (1994)), PompC (see, e.g., Bullifent et al., Vacccine, 18:2668-2676 (2000)), PnirB (see, e.g., Chatfield et al., Biotech. (NY), 10:888-892 (1992)), PssrA (see, e.g., Lee et al., J. Bacteriol. 182:771-781 (2000)), PproU (see, e.g., Rajkumari and Gowrishankar, J. Bacteriol., 183:6543-6550 (2001)), Pdps (see, e.g., Marshall et al., Vaccine, 18: 1298-1306 (2000)), and PssaG (see, e.g., McKelvie et al., Vaccine, 22:3243-3255 (2004)).

Some promoters are known to be regulated promoters that require the presence of some kind of activator or inducer molecule in order to transcribe a coding sequence to which they are operably linked. However, some promoters may be regulated or inducible promoters in E. coli, but function as unregulated promoters in Salmonella. An example of such a promoter is the well known trc promoter (“Ptrc”, see, e.g., Amman et al., Gene, 25(2-3):167-178 (1983)). As with Plac and Ptac, Ptrc functions as an inducible promoter in Escherichia coli (e.g., using the inducer molecule isopropyl-β-D-thio-galactopyranoside, “IPTG”), however, in Salmonella bacteria having no LacI repressor, Ptrc is an efficient constitutive promoter that readily transcribes plague antigen-containing polypeptide coding sequences present on antigen-expressing plasmids described herein. Accordingly, such a constitutive promoter does not depend on the presence of an activator or inducer molecule to express an antigen-containing polypeptide in a strain of Salmonella.

The plague antigen-expressing plasmids that reside in the live vaccine strains also contain an origin of replication (ori) that enables the plasmids to be maintained as multiple copies in the bacterial cell. A number of multi-copy plasmids that replicate in Salmonella bacteria are known in the art, as are various origins of replications for maintaining multiple copies of plasmids. Preferred origins of replications for use in the multi-copy antigen-expressing plasmids described herein include the origin of replication from the multi-copy plasmid pBR322 (“pBR ori”; see, e.g., Maniatis et al., In Molecular Cloning: A Laboratory Manual (Cold Spring Harbor Laboratory, Cold Spring Harbor, 1982), pp. 479-487; Watson, Gene, 70:399-403, 1988) and the origin of replication of pUC plasmids (“pUC ori”), such as found on plasmid pUC18 (see, e.g., Yanish-Perron et al., Gene, 33:103-119 (1985)).

Owing to the high degree of genetic identity and homology, any serovar of S. enterica may be used as the bacterial host for a live vaccine composition for plague provided the necessary attenuating mutations and antigen-expressing plasmids as described herein are also employed. Accordingly, serovars of S. enterica that may be used in the invention include those selected from the group consisting of Salmonella enterica serovar Typhimurium (“S. typhimurium”), Salmonella enterica serovar Typhi (“S. typhi”), Salmonella enterica serovar Paratyphi B (“S. paratyphi B”), Salmonella enterica serovar Paratyphi C (“S. paratyphi C”), Salmonella enterica serovar Hadar (“S. hadar”), Salmonella enterica serovar Enteriditis (“S. enteriditis”), Salmonella enterica serovar Kentucky (“S. kentucky”), Salmonella enterica serovar Infantis (“S. infantis”), Salmonella enterica serovar Pullorum (“S. pullorum”), Salmonella enterica serovar Gallinarum (“S. gallinarum”), Salmonella enterica serovar Muenchen (“S. muenchen”), Salmonella enterica serovar Anatum (“S. anatum”), Salmonella enterica serovar Dublin (“S. dublin”), Salmonella enterica serovar Derby (“S. derby”), and Salmonella enterica serovar Choleraesuis var. kunzendorf (“S. cholerae kunzendorf”).

Examples of attenuated strains of Salmonella that are useful in orally administrable, live vaccine compositions of the invention for protection against plague include the following strains of S. typhimurium that were deposited with the American Type Culture Collection (“ATCC”, 10801 University Blvd., Manassas, Va., 20110-2209, USA) under the terms of the Budapest Treaty on Dec. 2, 2004:

S. typhimurium strain M020 (ATCC Accession No. PTA-6406) that carries the antigen-expressing plasmid pMEG-1621 (see, FIG. 1) and that expresses an F1-V fusion polypeptide,

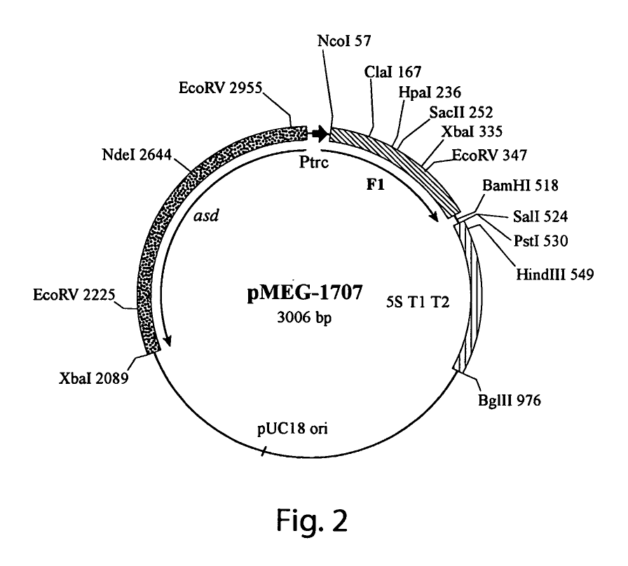

S. typhimurium M022 (ATCC Accession No. PTA-6407) that carries the antigen-expressing plasmid pMEG-1707 (see, FIG. 2) and that expresses an F1 antigen polypeptide,

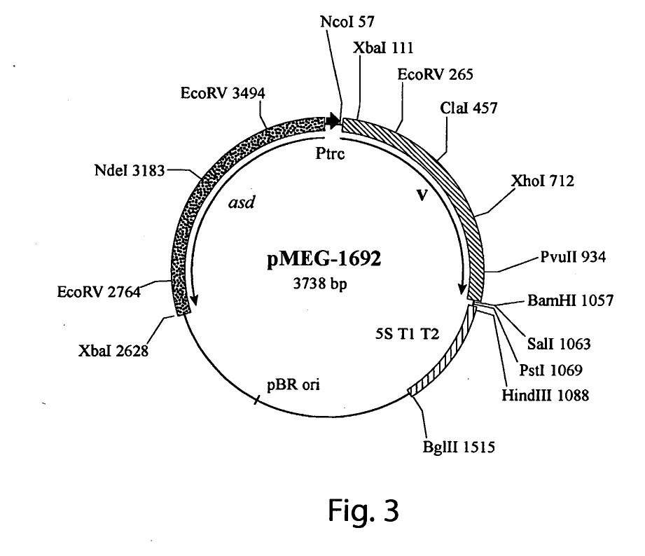

S. typhimurium M023 (ATCC Accession No. PTA-6408) that carries the antigen-expressing plasmid pMEG-1692 (see, FIG. 3) and that expresses a V antigen polypeptide,

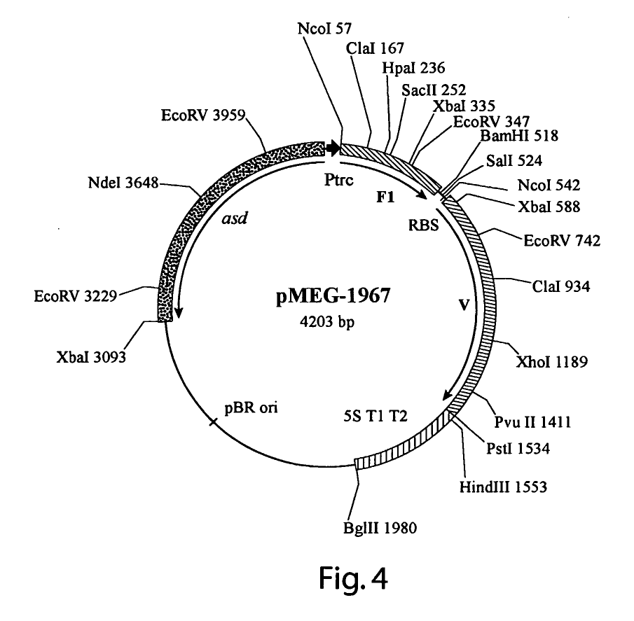

S. typhimurium M048 (ATCC Accession No. PTA-6409) that carries the antigen expressing-plasmid pMEG-1967 (see, FIG. 4) and that expresses an F1 antigen polypeptide and a V antigen polypeptide, and

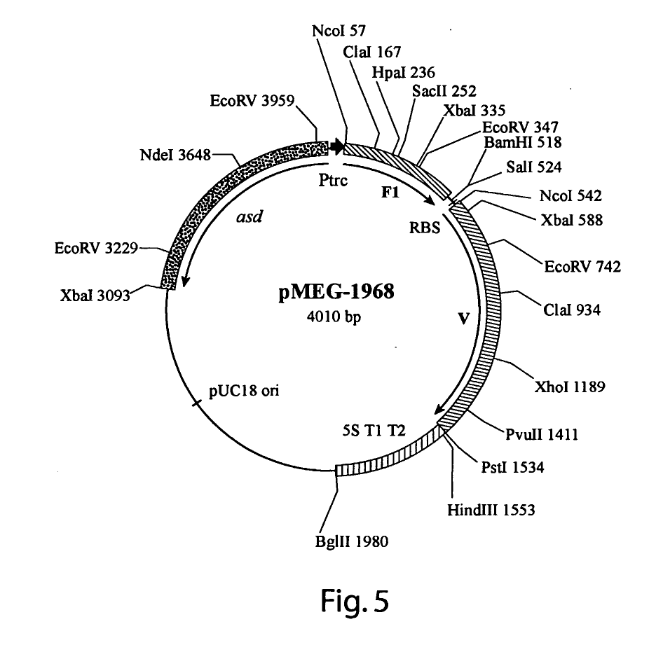

S. typhimurium M049 (ATCC Accession No. PTA-6410) that carries the antigen-expressing plasmid pMEG-1968 (see, FIG. 5) and that expresses an F1 antigen polypeptide and a V antigen polypeptide.

The vaccine compositions described herein may be administered orally to an individual in any form that permits the Salmonella bacterial strain of the composition to remain alive and to persist in the gut for a time sufficient to elicit an immune response to one or more plague antigens of Yersinia pestis expressed in the Salmonella strain. For example, the live bacterial strains described herein may be administered in relatively simple buffer or saline solutions at physiologically acceptable pH and ion content. By “physiologically acceptable” is meant whatever is compatible with the normal functioning physiology of an individual who is to receive a live vaccine composition described herein. Preferably, bacterial strains described herein are suspended in otherwise sterile solutions of bicarbonate buffers, phosphate buffered saline (PBS), or physiological saline, that can be easily swallowed by most individuals. However, “oral” routes of administration may include not only swallowing from the mouth a liquid suspension or solid form comprising a live bacterial strain described herein, but also administration of a suspension of a bacterial strain through a nasojejunal or gastrostomy tube, and rectal administration, e.g., by using a suppository comprising a live bacterial strain described herein to establish an infection by such bacterial strain in the lower intestinal tract of the alimentary canal. Accordingly, any of a variety of alternative modes and means may be employed to administer a vaccine composition described herein to the alimentary canal of an individual if the individual cannot swallow from the mouth.

In order to more fully illustrate the invention, the following non-limiting examples are provided.

### EXAMPLES

### Example 1

### Materials and Methods for Studies on Live Bacterial Vaccines for Plague

Materials for the preparation of standard growth media were obtained from Becton Dickinson Microbiology (Cockeysville, Md., USA) and prepared following manufacturer's instructions. The enzymes used in DNA manipulations were obtained from New England Biolabs and used according to manufacturer's instructions. Diaminopimelic acid (DAP) was commercially obtained (Sigma Chemical Co., St. Louis, Mo., USA).

The Escherichia coli and attenuated “Salmonella enterica subspecies enterica” serovar Typhimurium (S. typhimurium) bacterial strains used in the studies described below are listed in Table 1. Strains were grown at 37° C. in Luria broth supplemented with DAP (50 μg/ml) as needed.

TABLE 1
Bacterial Strains

| Bacterial Strain | Genotype | Plasmid | Antigen Expressed |
|---|---|---|---|
| Escherichia coli | | | |
| MGN-055 | φ80d lacZ ΔM15 deoR Δ(lacZYA- | pYA232 | LacI repressor |
| | argF)U169 supE44 λ-gyrA96 | | plasmid host |
| | recA1 relA1 endA1 Δ asdA4 | | |
| | Δzhf-2::Tn10 hsdR17 (R− M+) | | |
| Salmonella | | | |
| typhimurium | | | |
| MGN5760 | ΔphoP/Q956, ΔasdA19 | | Attenuated host |
| | (pBAD.C2) | | |
| M019 | ΔphoP/Q956, ΔasdA19 | pYA3342 | Vector only |
| | (pBAD.C2) | | |
| M020 | ΔphoP/Q956, ΔasdA19 | pMEG- | F1-V |
| | (pBAD.C2) | 1621 | |
| M022 | ΔphoP/Q956, ΔasdA19 | pMEG- | F1 |
| | (pBAD.C2) | 1707 | |
| M023 | ΔphoP/Q956, ΔasdA19 | pMEG- | V |
| | (pBAD.C2) | 1692 | |
| M048 | ΔphoP/Q956, ΔasdA19 | pMEG- | F1 and V from pBR |
| | (pBAD.C2) | 1967 | |
| M049 | ΔphoP/Q956, ΔasdA19 | pMEG- | F1 and V from pUC |
| | (pBAD.C2) | 1968 | |

The parent strain for the S. typhimurium isolates described herein is strain MGN-5760 that was created from S. typhimurium (ATCC Accession No. 14028, Manassas, Va., USA), a strain commonly used to study Salmonella pathogenesis. The first step in the construction of the bacterial vaccine strains described herein involved introduction of a deletion mutation in the phoP/Q virulence regulon to attenuate virulence (conducted by Elizabeth Hohmann, M.D., Massachusetts General Hospital, Boston, Mass., USA) that resulted in the ΔphoP/Q strain LH430 as previously described by Hohmann et al. (Vaccine. 14(1):19-24 (1996)). Briefly, a DNA fragment containing the entire phoP/Q locus was amplified by polymerase chain reaction (PCR) from S. typhimurium LT2 chromosomal DNA and subcloned into a high copy number vector, designated pLH356. Sequence data and restriction mapping of the phoP/Q locus revealed four internal HpaI restriction endonuclease sites. A deletion within the phoP/Q locus was made by digesting pLH356 with HpaI. The digested plasmid was re-ligated to yield a plasmid that contained a truncated phoP/Q locus lacking a 1203 base pair (bp) DNA segment between two HpaI sites. The 1203 bp deleted phoP/Q locus (designated ΔphoP/Q956) was verified by restriction enzyme digest analysis, and the plasmid was designated pLH418. A DNA fragment containing this ΔphoP/Q was isolated from pLH418 and subcloned into the suicide vector pCVD442 (Miller and Mekalanos, Proc. Natl. Acad. Sci. (USA), 86(13):5054-5058 (1988); Donnenberg and Kaper, Infect. Immun., 59(12):4310-4317 (1991)) to yield pLH423. The suicide vector, pCVD442, contains the pir-dependent R6K origin of replication and is not maintained in cells that lack the pir gene. Plasmid pCVD442 is maintained in the permissive host Escherichia coli SM10λpir. Mobilization of pCVD442-based plasmids into other Gram-negative bacteria (like Salmonella) is possible due to the presence of the mob region. The vector also encodes for ampicillin resistance and contains the sacB gene of Bacillus subtilis. pLH423 was transformed into E. coli SM10λpir and then moved into S. typhimurium ATCC 14028 by conjugal mating. Through the process of allelic exchange, the native phoP/Q allele on the Salmonella chromosome was replaced with the deleted allele (ΔphoP/Q956).

Each Salmonella strain carries an inactivated asd chromosomal gene. The introduction of this mutation produced strains suitable for use as balanced-lethal hosts that maintain the antigen-expressing plasmids described herein. For M020, an asd balanced-lethal system was developed in LH430 to support the maintenance of a recombinant plasmid expressing the F1-V fusion polypeptide. A deletion in the S. typhimurium LH430 gene encoding asd was introduced through the genetic process of allelic exchange, employing the pCVD442-based suicide plasmid, pMEG-611 (bearing the mutant asdA19 allele). The Salmonella strain bearing the asdA19 allele, designated MGN-5760, only grows in the presence of an exogenous source of diaminopimelic acid (DAP) or when transformed with a complementing Asd⁺ balanced-lethal plasmid.

The recombinant plasmid pPW731 that contains a coding sequence for an F1-V fusion polypeptide (see, SEQ ID NOs:5 and 6) was obtained from DynPort Vaccine Company (Frederick, Md., USA).

The plasmid pYA3341 is a colE1 replicon pUC18-based plasmid that encodes a promoterless, but otherwise wild type copy of the Salmonella asd gene and was created in the laboratory of Roy Curtiss III, at Washington University (St. Louis, Mo., USA).

The plasmid pYA3342 is a colE1 replicon, pBR322-based plasmid that encodes a promoterless, but otherwise wild type copy of the Salmonella asd gene and was created in the laboratory of Roy Curtiss III, at Washington University (St. Louis, Mo., USA).

When expressed in a compatible bacterial host, the asd coding sequence from plasmid pYA3341 or plasmid pYA3342 (yielding a wildtype asparate semialdehyde dehydrogenase) will complement the asd mutation in the chromosome of Asd⁻ strains, such as Escherichia coli strain MGN-055, allowing such strains to grow in the absence of an exogenous source of diaminopimelic acid (DAP).

Polyclonal antiserum that binds F1-V fusion polypeptide was obtained from male white New Zealand rabbits that were initially inoculated with recombinant F1-V fusion polypeptide (expressed from plasmid pPW73 1) in Complete Freund's Adjuvant (Sigma Chemical Co., St. Louis, Mo., USA) and subsequently boosted with F1-V fusion polypeptide in Incomplete Freund's Adjuvant (Sigma Chemical Co.).

Enzyme-linked immunosorbent assays (ELISAs) were performed by first coating plates (e.g., multi-welled microtiter plates) with recombinant F1-V, F1, or V polypeptide antigen (obtained from DynPort Vaccine Company) that was diluted to 1.0 μg/ml in 0.05 M carbonate buffer to give a final concentration of 0.1 μg per well. Coating antigen was allowed to bind to plates overnight at 4° C. Plates were then washed with phosphate buffered saline (“PBS”)/0.05% Tween detergent. A solution of 2.5% bovine serum albumin (“BSA”) in PBS was applied to the wells for 1 hour at 37° C. to block non-specific adsorption during the assay. Plates were washed and 200 μl of each serum sample, diluted 1:100 in 1% BSA/PBS, was added to the first column of wells while 100 μl of 1% BSA/PBS was added to all remaining wells. The samples were then serially diluted by pipetting 100 μl of sample from the first column to the second column, mixing, and repeating to the next column and continuing. Plates were incubated for 1 hour at 37° C., then washed. One hundred (100) μl of alkaline phosphatase-conjugated, goat anti-mouse IgG (KPL, Gaithersburg, Md., USA), diluted 1:500 in 1% BSA/PBS, was added to each well and incubated for 1 hour at 37° C. Plates were washed and developed using a 5-bromo-4-chloro-3-indoyl phosphate substrate for alkaline phosphatase as provided in the BluePhos® substrate Kit (KPL). Substrate reaction (color development) was stopped after 10 minutes with 2.5% EDTA tetrasodium salt, and the plates were read at 630 nm with a spectrophotometer.

### Example 2

### Construction and Characterization of an Attenuated Salmonella Bacterial Strain that Expresses an F1-V Fusion Polypeptide from a pBR322-Based, Antigen-Expressing Plasmid

The following study provided an attenuated Salmonella bacterial strain carrying an antigen-expressing plasmid that comprises a nucleotide sequence of SEQ ID NO:5 that encodes an F1-V fusion polypeptide having an amino acid sequence of SEQ ID NO:6.

Strain Construction

The coding region for the F1-V fusion protein in the recombinant plasmid pPW731 (DynPort Vaccine Company, Frederick, Md., USA) was amplified by polymerase chain reaction (PCR) amplified using the following primers:

| | |
|---|---|
| Primer F1-V.asd.F: | |
| 5′ TACATCCATGGCAGATTTAACTGCAAGC 3′ | (SEQ ID NO:7) |
| and | |
| Primer F1-V.asd.R: | |
| 5′ CGCGGATCCTCATTTACCAGACGTGTCATC 3′. | (SEQ ID NO:8) |

The F1-V PCR product (PCR amplicon) so obtained and the Asd⁺ plasmid, pYA3342, were then digested with restriction endonucleases NcoI and BamHI and the digestion products purified using the Qiaquick® PCR Purification Kit (Qiagen, Inc., Valencia, Calif., USA). The purified DNA fragments were joined using T4 DNA ligase (New England Biolabs, Beverly, Mass., USA), and electroporated into the E. coli strain MGN-055. Isolated colonies capable of growing without DAP were screened by PCR for the expected F1-V insert fragment and for the presence of a 4178 base pair (bp) plasmid using QiaPrep® Spin MiniPrep Kits (Qiagen). Plasmid DNA content was determined by agarose gel electrophoresis. Isolates that yielded the expected F1-V PCR product and that possessed plasmids of the correct size were further analyzed for expression of the desired F1-V fusion polypeptide by polyacrylamide gel electrophoresis (PAGE) and Western immunoblot analysis using an anti-F1-V specific polyclonal rabbit serum. One of the isolates expressed a protein of the expected size for the F1-V fusion polypeptide (approximately 53,000 daltons) that reacted with the F1-V specific antisera on immunoblots. This isolate contained a plasmid that was designated pMEG-1621. Plasmid pMEG-1621 contains a strong constitutive (i.e., in Salmonella) promoter, Ptrc, driving the transcription of the F1-V coding region, followed by a 5S rRNA T1 T2 transcription terminator to reduce interference with plasmid replication (see, FIG. 1). The plasmid pMEG-1621 was electroporated into S. typhimurium MGN-5760 (ΔphoP/Q956, ΔasdA19 (PBAD.C2)), and the expression of the F1-V fusion polypeptide confirmed by PAGE and Western immunoblot analysis using the F1-V specific polyclonal rabbit antiserum. The results indicated that the F1-V fusion polypeptide was encoded on and expressed from plasmid pMEG-1621 resident in the isolated bacterial strain. The isolate was cell banked as strain M020.

Evaluating the Immunogenicity of M020 Following Oral Immunization of BALB/c Mice

The immunogenicity of M020 was evaluated in BALB/c mice. Briefly, ten (10) 6-week-old, female, BALB/c mice were orally administered (“vaccinated”) by pipette-feeding with one priming dose of 1×10⁹ colony-forming units (“cfu”) of S. typhimurium strain M020 on Day 1, followed by an “oral booster vaccination” of 1×10⁹ cfu by pipette feeding on Day 14. Blood samples were collected on Days-2 (prior to the vaccination) and again following the booster immunization on Days 28 and 42. Table 2, below, summarizes the immunogenicity data from this experiment.

TABLE 2
Reciprocal Antibody Titers Elicited by M020 in BALB/c Mice*

| Serum IgG | | Serum IgG | | Serum IgG | |
|---|---|---|---|---|---|
| Anti-F1 | | Anti-V | | Anti-F1-V | |
| 2 weeks | 4 weeks | 2 weeks | 4 weeks | 2 weeks | 4 weeks |
| post-boost | post-boost | post-boost | post-boost | post-boost | post-boost |
| 137 | 467 | 685 | 1008 | 2341 | 2017 |

*Values are geometric means from ten mice

The results in Table 2 indicate that mice that were administered M020 expressing the F1-V fusion polypeptide developed antibody responses against the F1 antigen, the V antigen, and the F1-V fusion polypeptide.

### Example 3

### Construction and Characterization of an Attenuated Salmonella Bacterial Strain that Expresses an F1 Antigen Polypeptide from a pUC18 -Based Antigen-Expressing Plasmid

The following study provided an attenuated Salmonella bacterial strain carrying an antigen-expressing plasmid that has an origin of replication from plasmid pUC18 and that comprises a nucleotide sequence of SEQ ID NO:1 that encodes an F1 antigen polypeptide having an amino acid sequence of SEQ ID NO:2.

Strain Construction

The coding region for the F1 protein was PCR amplified from the recombinant plasmid pPW731 obtained from DynPort Vaccine Company using the following primers:

Primer F1.asd.F:

(SEQ ID NO:9)

5′ TACATGCCATGGCAGATTTAACTGCAAGC 3′
Primer F1.asd.R:

(SEQ ID NO:10)

5′ CGCGGATCCTTATTGGTTAGATACGGTTACG 3′.

The F1 PCR product so obtained and the pUC-based Asd⁺ plasmid, pYA3341, were digested with restriction endonucleases NcoI and BamHI, and the digestion products purified using the Qiaquick® PCR Purification Kit (Qiagen). Purified DNA fragments were joined using T4 DNA ligase and electroporated into the E. coli strain MGN-055. Isolated colonies capable of growing without DAP were screened by PCR for the expected F1 insert fragment and for the presence of a 3006 bp plasmid using QiaPrep® Spin MiniPrep Kits (Qiagen). Plasmid DNA content was determined by agarose gel electrophoresis. Isolates that yielded the expected F1 PCR product and that possessed plasmids of the correct size were further analyzed for expression of the desired F1 polypeptide by PAGE and Western immunoblot using the F1-V specific polyclonal rabbit antiserum described above. One of these isolates expressed a protein of the expected size for the F1 polypeptide (approximately 16,000 daltons) that reacted with the F1-V specific antiserum on immunoblots. This isolate contained a plasmid that was designated pMEG-1707. Plasmid pMEG-1707 contains the strong constitutive promoter, Ptrc, driving the transcription of the F1 coding region, followed by a 5S rRNA T1 T2 transcription terminator to reduce interference with plasmid replication (see, FIG. 2). The plasmid pMEG-1707 was electroporated into S. typhimurium MGN-5760 (Δphop/Q956, ΔasdA19 (pBAD.C2)) and confirmed by PAGE and Western immunoblot analysis to express a protein of the expected size for the F1 polypeptide that reacts with the F1-V specific polyclonal rabbit antiserum. The results indicated that the F1 polypeptide was encoded on and expressed from plasmid pMEG-1707 resident in the isolated bacterial strain. The isolate was cell banked as strain M022.

### Example 4

### Construction and Characterization of an Attenuated Salmonella bacterial Strain that Expresses a V Antigen Polypeptide from a pBR322-Based, Antigen-Expressing Plasmid

The following study provided an attenuated Salmonella bacterial strain carrying an antigen-expressing plasmid that has an origin of replication from plasmid pBR322 and that comprises a nucleotide sequence of SEQ ID NO:3 that encodes a V antigen polypeptide having an amino acid sequence of SEQ ID NO:4.

Strain Construction

The coding region for the V protein was PCR amplified from the recombinant plasmid pPW731 obtained from DynPort Vaccine Company using the following primers:

Primer V.asd.F:

(SEQ ID NO:11)

5′ TACATGCCATGGTTAGAGCCTACGAAC 3′
and
Primer V.asd.R:

(SEQ ID NO:12)

5′ CGCGGATCCTCATTTACCAGACGTGTCATC 3′.

The V PCR product so obtained and the Asd+ plasmid, pYA3342, were digested with restriction endonucleases NcoI and BamHI, and the digestion products purified using the Qiaquick® PCR Purification Kit (Qiagen). Purified DNA fragments were joined using T4 DNA Ligase (New England Biolabs) and electroporated into the E. coli strain MGN-055. Isolated colonies capable of growing without DAP were screened by PCR for the expected V insert fragment and for the presence of a 3738 bp plasmid using QiaPrep® Spin MiniPrep Kits (Qiagen). Plasmid DNA content was determined by agarose gel electrophoresis. Isolates that yielded the expected V PCR product and that possessed plasmids of the correct size were further analyzed by PAGE and Western immunoblot analysis using the F1-V specific polyclonal rabbit antiserum described above. One of these isolates expressed a protein of the expected size for the V polypeptide (approximately 37,000 daltons) that reacted with the F1-V specific rabbit antiserum on immunoblots. This isolate contained a plasmid that was designated pMEG-1692. Plasmid pMEG-1692 contains the strong constitutive promoter, Ptrc, driving the transcription of the V coding region, followed by a 5S rRNA T1 T2 transcription terminator to reduce interference with plasmid replication (see, FIG. 3). The plasmid pMEG-1692 was electroporated into S. typhimurium MGN-5760 (Δphop/Q956, ΔasdA19 (PBAD.C2)) and confirmed by PAGE and Western immunoblot analysis to express a protein of the expected size for the V polypeptide that reacts with the F1-V specific polyclonal rabbit antiserum. The results indicated that the V antigen polypeptide was encoded on and expressed from plasmid MEG-1692 resident in the isolated strain. The isolate was cell banked as strain M023

### Example 5

### Construction and Characterization of an Attenuated Salmonella Bacterial Strain that Expresses an F1 Antigen Polypeptide and a V Antigen Polypeptide from a pBR322-Based, Antigen-Expressing Plasmid

The following study provided an attenuated Salmonella bacterial strain carrying an antigen-expressing plasmid that has an origin of replication from plasmid pBR322 and that comprises a nucleotide sequence of SEQ ID NO:1 that encodes an F1 antigen polypeptide having an amino acid sequence of SEQ ID NO:2 and a nucleotide sequence of SEQ ID NO:3 that encodes a V antigen polypeptide having an amino acid sequence of SEQ ID NO:4.

Strain Construction

The coding region for the V protein was PCR amplified from the V Asd+ plasmid, pMEG-1692 (see, above) using the following primers:

Primer RBS + V.F.Sal:

(SEQ ID NO:13)

5′ ACGCGTCGACACAGGAAACAGACCATGGTTAGAGCCTAC 3′
and
Primer V.R.Pst

(SEQ ID NO:14)

5′ AAAACTGCAGTCATTTACCAGACGTGTCATC 3′.

The V PCR product so obtained and the pBR322-based F1 Asd+ plasmid, pMEG-1702, were then digested with restriction endonucleases SalI and PstI, and the digestion products purified using the Qiaquick® PCR Purification Kit (Qiagen). The purified DNA fragments were joined using T4 DNA ligase (New England Biolabs) and electroporated into the E. coli strain MGN-055. Isolated colonies capable of growing without DAP were screened by PCR for the expected V insert fragment and for the presence of a 4203 bp plasmid using QiaPrep® Spin MiniPrep Kits (Qiagen). Plasmid DNA content was determined by agarose gel electrophoresis. Isolates that yielded the expected V PCR product and that possessed plasmids of the correct size were further analyzed for expression of F1 and V antigen polypeptides by PAGE and Western immunoblot using the F1-V specific polyclonal rabbit antiserum. One of these isolates expressed proteins of the expected size for the F1 polypeptide (approximately 16,000 daltons) and for the V polypeptide (approximately 37,000 daltons) that reacted with the F1-V specific antiserum. This isolate contained a plasmid that was designated pMEG-1967. Plasmid pMEG-1967 contains the strong constitutive promoter, Ptrc, driving the transcription of an operon consisting of the F1 coding region and a V coding region, each with its own ribosomal binding site (RBS) to allow translation of the separate F1 and V coding sequences (present on a single mRNA transcript) into the corresponding and separate F1 and V polypeptides (see, FIG. 4). The plasmid pMEG-1967 was electroporated into S. typhimurium MGN-5760 (Δphop/Q956, ΔasdA19 (pBAD.C2)) and confirmed by PAGE and western immunoblot analysis to express proteins of the expected size for the F1 antigen polypeptide and the V antigen polypeptide that reacted with the F1-V specific polyclonal rabbit antiserum. The results indicated that the F1 and V antigen polypeptides were encoded on and expressed from plasmid MEG-1967 resident in the isolated strain. The isolate was cell banked as strain M048.

### Example 6

### Construction and Characterization of an Attenuated Salmonella Bacterial Strain that Expresses an F1 Antigen Polypeptide and a V Antigen Polypeptide from a pUC18-Based, Antigen-Expressing Plasmid

The following study provided an attenuated Salmonella bacterial strain carrying an antigen-expressing plasmid that has an origin of replication from plasmid pUC18 and that comprises a nucleotide sequence of SEQ ID NO:1 that encodes an F1 antigen polypeptide having an amino acid sequence of SEQ ID NO:2 and a nucleotide sequence of SEQ ID NO:3 that encodes a V antigen polypeptide having an amino acid sequence of SEQ ID NO:4.

Strain Construction

The coding region for the V protein was PCR amplified from the V Asd⁺ plasmid, pMEG-1692, using the following primers:

Primer RBS + V.F.Sal:

(SEQ ID NO:13)

5′ ACGCGTCGACACAGGAAACAGACCATGGTTAGAGCCTAC 3′
and
Primer V.R.Pst:

(SEQ ID NO:14)

5′ AAAACTGCAGTCATTTACCAGACGTGTCATC 3′.

The V PCR product so obtained and the pUC-based F1 Asd⁺ plasmid, pMEG-1707, were digested with restriction endonucleases SalI and PstI, and the digestion products purified using the Qiaquick® PCR Purification Kit (Qiagen). The purified DNA fragments were joined using T4 DNA ligase (New England Biolabs) and electroporated into the E. coli strain MGN-055. Isolated colonies capable of growing without DAP were screened by PCR for the expected V insert fragment and for the presence of a 4010 bp plasmid using QiaPrep® Spin MiniPrep Kits (Qiagen). Plasmid DNA content determined by agarose gel electrophoresis. Isolates that yielded the expected V PCR product and that possessed plasmids of the correct size were further analyzed for expression of F1 and V antigen polypeptides by PAGE and Western immunoblot analysis using the F1-V specific polyclonal rabbit antiserum. One of the isolates expressed proteins of the expected size for the F1 polypeptide (approximately 16,000 daltons) and for the V polypeptide (approximately 37,000 daltons) that reacted with the F1-V specific antiserum on immunoblots. This isolate contained a plasmid that was designated pMEG-1968. Plasmid pMEG-1968 contains the strong constitutive promoter, Ptrc, driving the transcription of an operon consisting of the F1 coding region and a V coding region, each with its own ribosomal binding site (RBS) to allow translation of the separate F1 and V coding sequences (present on a single mRNA transcript) into the corresponding and separate F1 and V polypeptides (see, FIG. 5). The plasmid pMEG-1968 was electroporated into S. typhimurium MGN-5760 (Δphop/Q956, ΔasdA19 (pBAD.C2)) and confirmed by PAGE and Western immunoblot analysis to express proteins of the expected size for the F1 polypeptide and the V polypeptide that reacts with the F1-V specific polyclonal rabbit antiserum. The results indicated that the F1 and V antigen polypeptides were encoded on and expressed from plasmid MEG-1968 resident in the isolated strain. The isolate was cell banked as strain M049.

### Example 7

### Evaluation of the Immunogenicity of Live Plague Vaccines Strains Following Oral Administration to BALB/c Mice

The immunogenicity of strains M019 (vector only control), M020 (expressing an F1-V fusion polypeptide), M022 (expressing an F1 antigen polypeptide) M023 (expressing a V antigen polypeptide), M048 (expressing F1 and V antigen polypeptides) and M049 (expressing F1 and V antigen polypeptides) were evaluated in BALB/c mice. Briefly, two groups of five, 6-week old, female, BALB/c mice were orally administered (“vaccinated”) by pipette-feeding one priming dose of 1×10⁹ cfu of each S. typhimurium strain (on Day 1) followed by an “oral booster vaccination” of 1×10⁹ cfu by pipette feeding on Day 14. Blood samples were collected on Day-2 (prior to the vaccination) and again following the booster vaccination (“post-boost”) on Days 28 and 42. Table 3, below, summarizes the immunogenicity data from this experiment.

TABLE 3
Average Reciprocal Antibody Titers Elicited by Plague Vaccines in BALB/c Mice

| | Serum IgG | | Serum IgG | | Serum IgG | |
|---|---|---|---|---|---|---|
| Strain (expressed | Anti-F1 | | Anti-V | | Anti-F1-V | |
| antigenic | 2 weeks | 4 weeks | 2 weeks | 4 weeks | 2 weeks | 4 weeks |
| polypeptide) | post-boost | post-boost | post-boost | post-boost | post-boost | post-boost |
| M019 | <100 | <100 | <100 | <100 | <100 | <100 |
| (control) | | | | | | |
| M020 | 220 | 80 | 1140 | 1200 | 3540 | 3440 |
| (F1-V) | | | | | | |
| M022 | 700 | 2320 | N/A | N/A | 700 | 2480 |
| (F1) | | | | | | |
| M023 | N/A | N/A | 2080 | 14,400 | 8320 | 34,560 |
| (V) | | | | | | |
| M048 | N/A | <100 | N/A | 9680 | N/A | 38,720 |
| (F1 + V) | | | | | | |
| M049 | N/A | 3000 | N/A | 400 | N/A | 4000 |
| (F1 + V) | | | | | | |

N/A = not applicable

The data in Table 3 show that vaccinated mice developed antibody responses against the F1 antigen polypeptide, the V polypeptide, and the F1-V fusion polypeptide. Strains that contain a pUC18-based, F1-expressing plasmid (i.e., strains M022 and M049) induced the best immune responses to F1, however, strain M049 that contains a pUC18-based, F1 and V antigen-expressing plasmid elicited a weaker immune response to the V antigen relative to strain M048 that carries the analogous, pBR322-based, F1 and V antigen-expressing plasmid.

All patents, applications, and publications cited in the above text are incorporated herein by reference.

Other variations and embodiments of the invention described herein will now be apparent to those of ordinary skill in the art without departing from the scope of the invention or the spirit of the claims below.

1. A live vaccine composition for protecting against plague comprising a live attenuated bacterium that is a serovar of Salmonella enterica comprising:
an attenuating mutation in a genetic locus of the chromosome of said bacterium that attenuates virulence of said bacterium and wherein said attenuating mutation is not a single mutation in a gene that encodes a protein that is essential for the synthesis of an aromatic compound and is not a single mutation in a gene for galactose utilization;
a lethal mutation in a genetic locus in the chromosome of said bacterium wherein said lethal mutation prevents expression from said genetic locus of a protein that has an activity that is essential for cell wall synthesis of said bacterium;
an antigen-expressing, multi-copy plasmid comprising:
a nucleotide sequence coding for an immunogenic polypeptide comprising a Yersinia pestis V antigen, an immunogenic portion of said V antigen, a Yersinia pestis F1 antigen, an immunogenic portion of said F1 antigen, or a combination thereof, wherein said nucleotide sequence is operably linked to a promoter that permits intracellular expression of said immunogenic polypeptide from said plasmid,
a gene encoding a protein that has an activity that is essential for cell wall synthesis, wherein expression of said protein essential for cell wall synthesis complements said lethal mutation in the chromosome of said bacterium and thereby permits growth of said bacterium, and
an origin of replication that permits multiple copies of said plasmid to be maintained in said bacterium,
wherein said live vaccine composition elicits an immune response to one or more Yersinia pestis antigen when administered orally to an individual.

2. The live vaccine composition according to claim 1, wherein said serovar of S. enterica is selected from the group consisting of Salmonella enterica serovar Typhimurium (S. typhimurium), Salmonella enterica serovar Typhi (S. typhi), Salmonella enterica serovar Paratyphi B (S. paratyphi B), Salmonella enterica serovar Paratyphi C (S. paratyphi C), Salmonella enterica serovar Hadar (S. hadar), Salmonella enterica serovar Enteriditis (S. enteriditis), Salmonella enterica serovar Kentucky (S. kentucky), Salmonella enterica serovar Infantis (S. infantis), Salmonella enterica serovar Pullorum (S. pullorum), Salmonella enterica serovar Gallinarum (S. gallinarum), Salmonella enterica serovar Muenchen (S. muenchen), Salmonella enterica serovar Anatum (S. anatum), Salmonella enterica serovar Dublin (S. dublin), Salmonella enterica serovar Derby (S. derby), and Salmonella enterica serovar Choleraesuis var. kunzendorf.

3. The live vaccine composition according to claim 2, wherein said serovar of S. enterica is S. enterica serovar Typhimurium (S. typhimurium).

4. The live vaccine composition according to claim 1, wherein said attenuating mutation is in a genetic locus selected from the group consisting of phoP, phoQ, cdt, cya, crp, poxA, rpoS, htrA, nuoG, pmi, galE, pabA, pts, damA, purB, gua, cadA, rfc, rfb, rfa, ompR, and combinations thereof.

5. The live vaccine composition according to claim 4, wherein said attenuating mutation is a deletion mutation.

6. The live vaccine composition according to claim 5, wherein said attenuating mutation is a ΔphoP/Q mutation.

7. The live vaccine composition according to claim 1, wherein said lethal mutation is a deletion in the asdA gene (ΔasdA) and said immunogenic polypeptide encoded on said antigen-expressing, multi-copy plasmid is a fusion protein comprising a V antigen or an immunogenic portion thereof, linked to an F1 antigen or an immunogenic portion thereof.

8. The live vaccine composition according to claim 1, wherein said origin of replication of said multi-copy plasmid is a pUC or pBR322 plasmid origin of replication.

9. The live vaccine composition according to claim 1 further comprising a physiologically acceptable buffer or saline solution.

10. A live vaccine composition comprising a live attenuated bacterium that is a Typhimurium serovar of Salmonella enterica selected from the group consisting of S. typhimurium strain M020 (ATCC Accession No. PTA-6406), S. typhimurium M022 (ATCC Accession No. PTA-6407), S. typhimurium M023 (ATCC Accession No. PTA-6408), S. typhimurium M048 (ATCC Accession No. PTA-6409), S. typhimurium M049 (ATCC Accession No. PTA-6410), and combinations thereof.

11. A live vaccine composition according to claim 10 further comprising a physiologically acceptable buffer or saline solution.

12. A method of protecting an individual from plague comprising administering to said individual a live vaccine composition according to claim 1 along the alimentary canal of said individual.

13. The method according to claim 12, wherein said live vaccine composition is administered to an individual by swallowing from the mouth, by a nasojejunal tube, by a gastrostomy tube, or by a suppository.

14. (canceled)

15. (canceled)


SEQUENCE LISTING (14 sequences)

SEQ ID NO: 1 - DNA, 453 bp, Yersinia pestis
atggcagatttaactgcaagcaccactgcaacggcaactcttgttgaaccagcccgcatcactcttacatataaggaaggcgctccaattacaattatggacaatggaaacatcgatacagaattacttgttggtacgcttactcttggcggctataaaacaggaaccactagcacatctgttaactttacagatgccgcgggtgatcccatgtacttaacatttacttctcaggatggaaataaccaccaattcactacaaaagtgattggcaaggattctagagattttgatatctctcctaaggtaaacggtgagaaccttgtgggggatgacgtcgtcttggctacgggcagccaggatttctttgttcgctcaattggttccaaaggcggtaaacttgcagcaggtaaatacactgatgctgtaaccgtaaccgtatctaaccaataa

SEQ ID NO: 2 - protein, 150 aa, Yersinia pestis
MADLTASTTATATLVEPARITLTYKEGAPITIMDNGNIDTELLVGTLTLGGYKTGTTSTSVNFTDAAGDPMYLTFTSQDGNNHQFTTKVIGKDSRDFDISPKVNGENLVGDDVVLATGSQDFFVRSIGSKGGKLAAGKYTDAVTVTVSNQ

SEQ ID NO: 3 - DNA, 981 bp, Yersinia pestis
atggttagagcctacgaacaaaacccacaacattttattgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatccatgataaatccattaatctcatggataaaaatttatatggttatacagatgaagagatttttaaagccagcgcagagtacaaaattctcgagaaaatgcctcaaaccaccattcaggtggatgggagcgagaaaaaaatagtctcgataaaggactttcttggaagtgagaataaaagaaccggggcgttgggtaatctgaaaaactcatactcttataataaagataataatgaattatctcactttgccaccacctgctcggataagtccaggccgctcaacgacttggttagccaaaaaacaactcagctgtctgatattacatcacgttttaattcagctattgaagcactgaaccgtttcattcagaaatatgattcagtgatgcaacgtctgctagatgacacgtctggtaaatga

SEQ ID NO: 4 - protein, 326 aa, Yersinia pestis
MVRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGK

SEQ ID NO: 5 - DNA, 1437 bp, Yersinia pestis
atggcagatttaactgcaagcaccactgcaacggcaactcttgttgaaccagcccgcatcactcttacatataaggaaggcgctccaattacaattatggacaatggaaacatcgatacagaattacttgttggtacgcttactcttggcggctataaaacaggaaccactagcacatctgttaactttacagatgccgcgggtgatcccatgtacttaacatttacttctcaggatggaaataaccaccaattcactacaaaagtgattggcaaggattctagagattttgatatctctcctaaggtaaacggtgagaaccttgtgggggatgacgtcgtcttggctacaggcagccaggatttctttgttcgctcaattggttccaaaggcggtaaacttgcagcaggtaaatacactgatgctgtaaccgtaaccgtatctaaccaagaattcatgattagagcctacgaacaaaacccacaacattttattgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatccatgataaatccattaatctcatggataaaaatttatatggttatacagatgaagagatttttaaagccagcgcagagtacaaaattctcgagaaaatgcctcaaaccaccattcaggtggatgggagcgagaaaaaaatagtctcgataaaggactttcttggaagtgagaataaaagaaccggggcgttgggtaatctgaaaaactcatactcttataataaagataataatgaattatctcactttgccaccacctgctcggataagtccaggccgctcaacgacttggttagccaaaaaacaactcagctgtctgatattacatcacgttttaattcagctattgaagcactgaaccgtttcattcagaaatatgattcagtgatgcaacgtctgctagatgacacgtctggtaaatga

SEQ ID NO: 6 - protein, 478 aa, Artificial
  F1-V Antigen Polypeptide
MADLTASTTATATLVEPARITLTYKEGAPITIMDNGNIDTELLVGTLTLGGYKTGTTSTSVNFTDAAGDPMYLTFTSQDGNNHQFTTKVIGKDSRDFDISPKVNGENLVGDDVVLATGSQDFFVRSIGSKGGKLAAGKYTDAVTVTVSNQEFMIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGK

SEQ ID NO: 7 - DNA, 28 bp, Artificial
  F1-V.asd.F
tacatccatggcagatttaactgcaagc

SEQ ID NO: 8 - DNA, 30 bp, Artificial
  Primer F1-V.asd.R
cgcggatcctcatttaccagacgtgtcatc

SEQ ID NO: 9 - DNA, 29 bp, Artificial
  Primer F1.asd.F
tacatgccatggcagatttaactgcaagc

SEQ ID NO: 10 - DNA, 31 bp, Artificial
  Primer F1.asd.R
cgcggatccttattggttagatacggttacg

SEQ ID NO: 11 - DNA, 27 bp, Artificial
  Primer V.asd.F
tacatgccatggttagagcctacgaac

SEQ ID NO: 12 - DNA, 30 bp, Artificial; identical to SEQ ID NO: 8
  Primer V.asd.R

SEQ ID NO: 13 - DNA, 39 bp, Artificial
  Primer RBS+V.F.Sal
acgcgtcgacacaggaaacagaccatggttagagcctac

SEQ ID NO: 14 - DNA, 31 bp, Artificial
  Primer V.R.Pst
aaaactgcagtcatttaccagacgtgtcatc